(19) United States
(12) Patent Application Publication
(10) Pub. No.: US 2010/0114011 A1
(43) Pub. Date: May 6, 2010

(54) METERING SYSTEM FOR OZONE OR OZONE/OXYGEN MIXTURE

(76) Inventor: Wolfgang Herrmann, Eichelsbach (DE)

Correspondence Address: KREMBLAS, FOSTER, PHILLIPS & POLLICK, 7632 SLATE RIDGE BOULEVARD, REYNOLDSBURG, OH, 43068, US

(21) Appl. No.: 12/374,736

(22) PCT Filed: Jul. 18, 2007
(86) PCT No.: PCT/EP2007/057437

§ 371 Date: Dec. 14, 2009

(30) Foreign Application Priority Data
Jul. 24, 2006 (EP) 06117737.4

(51) Int. Cl. A61M 13/00; G05D 11/00

(52) U.S. Cl. 604/25; 137/100

(57) ABSTRACT

Apparatus for preparing and/or delivering ozone or an ozone/oxygen mixture in metered amounts from oxygen, in particular for intraperitoneal administration into an animal or human body, having a fluid source or at least one fluid source connection (1) for providing oxygen, ozone or ozone/oxygen mixture and having a metering outlet (18), with a mass flow controller (5) being inserted into the fluid line from the fluid source connection (1) to the metering outlet (18) and making it possible to control the flow of fluid mass in accordance with a comparison of set value with actual value, with an operative connection existing or being able to exist between the inlet of the mass flow controller (5) and a fluid shut-off device (2) and between the metering outlet (18) and a delivery shut-off device (16), and with electronic control means (14) being connected at least to the two shut-off devices (2, 16) for control thereof.

### CROSS-REFERENCES TO RELATED APPLICATIONS

(Not Applicable)

This application is a submission to enter the national stage under 35 U.S.C. 371 for international application number PCT/EP2007/057437 having international filing date Jul. 18, 2007, for which priority was based upon patent application 06117737.4 having a filing date of Jul. 24, 2006 filed in Europe. The above prior application or applications are hereby incorporated by reference.

### STATEMENT REGARDING FEDERALLY-SPONSORED RESEARCH AND DEVELOPMENT

(Not Applicable)

### REFERENCE TO AN APPENDIX

(Not Applicable)

### BACKGROUND OF THE INVENTION

1. Field of the Invention

The invention concerns a device for preparing and delivering ozone or an ozone/oxygen mixture in metered amounts, wherein the ozone or ozone/oxygen mixture is made from oxygen. This device is suitable in particular for intraperitoneal administration into an animal or human body. The device has at least one connection for coupling to a fluid source, assuming such a fluid source is not provided directly in the device. The fluid source connection is used to provide oxygen, ozone or an ozone/oxygen mixture, depending upon whether ozone is supplied to the device from an external source or is produced internally in the device. The invention further concerns an apparatus for administration and/or insufflation of a fluid comprised of the ozone or an ozone/oxygen mixture, wherein said application apparatus is suitable for coupling to the device.

2. Description of the Related Art

The bactericidal effect of ozone and ozonized fluids has long been known in technical applications, and is used, for example, in the disinfection of drinking water in drinking water purification. Ozone is also used for medical applications, for example in hyperbaric ozone therapy. Recent research has also shown that treating tumors with ozone can produce excellent results. However, because ozone can also present a health hazard, the standards for safe administration, especially in terms of the amount and concentration of the ozone used, are high.

The German patent application 10 2004 017 599 discloses a device for the intraperitoneal administration of a fluid containing ozone or ozonized oxygen. This device has a fluid storage tank for storing the fluid to be administered, a high-precision manometer for measuring the pressure of administration, a device for delivering the fluid from the fluid storage tank, and a function for monitoring the pressure of the fluid being administered, which is coupled with a warning and switch-off function for overpressures. However, this device does not permit monitoring of additional parameters, such as ozone concentration or flow rate, for example. Moreover, when used for therapeutic applications, fluctuations in the pressure level must be balanced manually. The use of an ozone-fluid mixture prepared in advance, which must be placed in a storage container, and which is measured precisely according to the planned metering amount, results in the disadvantage that flexible reaction to changing requirements, circumstances and boundary conditions is impossible. In this respect, the design comprising the storage container which is precisely metered in advance is not adaptable. Ozone or ozone/fluid mixture that is not consumed or used remains in the storage container and, under the best circumstances, can be reused only with difficulty and at great expense.

U.S. Pat. No. 5,797,872 discloses a device for treating cow udders with ozone, having an ozone generator for generating ozone from oxygen or air, an ozone analyzer for determining the generated ozone concentration or density, a valve for controlling the flow rate, and a device for decomposing surplus ozone. However, this device is not suitable for intraperitoneal administration and has only a pressure meter with a shut-off function in the event of excess pressure.

In US-A-2002/0 055 706 a method for treating the human cervix, for example, using ozone is described. The treatment apparatus is equipped extensively with electronic control and monitoring means, for instance an electronic control system, which is coupled to a PC, a display, a memory and a multitude of sensors, i.a., for measuring temperature, pressure, Ph level, flow rate, gas concentration, etc., and which controls valves at the outlet side. The control system is especially coupled to a flow sensor in the ozone outlet line and an outlet valve for ozone, which is coupled at its input side to an ozone source and which functions as a control element. The controller itself is embodied in the control system, which also performs other functions.

From the company publication, “Was Sauerstoff nicht kann, vermag Ozon” [“What Oxygen Can't Do, Ozone Can”], published by the applicant in October 2003, an apparatus for use in ozone therapy in humans is known, with which incremental operator control using a touch screen is provided by means of a stored program control (SPC). The SPC also serves as the mechanism for controlling the ozone concentration, in that the SPC automatically controls measurements of the ozone concentration via an ozone analyzer and analog/digital converter, and uses a digital/analog converter to control an ozone generator in terms of control engineering as a control element. The metered amounts can also be adjusted precisely using a fluid storage container.

### BRIEF SUMMARY OF THE INVENTION

The object of the invention is to increase metering precision in an ozone delivery system. For the attainment of this object, refer to the device for preparing and/or delivering ozone or ozone/oxygen mixture disclosed in claim 1 and to the associated application set for connection to the metering outlet of the device, disclosed in claim 20. Advantageous, optional embodiments are disclosed in the dependent claims.

Because, in accordance with one characterizing feature of the invention, the mass flow controller is configured having its own intake for fluid flow to be controlled, independent of the electronic control system, and/or because this intake can be closed directly by a fluid shut-off device assigned specifically to it, which can be actuated via the control system, the pathway is cleared for an additional embodiment of the invention: The mass flow controller is created as a separate, compact structural unit, which performs the control with optimum precision independently of the electronic control system, using its own internal control circuit and set value/actual value comparison.

In accordance with another characterizing feature of the invention, the mass flow controller is especially spatially/structurally inserted in the flow path that leads through the device between the fluid source connection and the metering outlet. When combined with the further characterizing feature of the invention, according to which electronically controllable or actuable shut-off devices for the fluid flow are provided both upstream from the intake and downstream from the outlet of the mass flow controller in the direction of fluid flow, the synergistic effect is produced that, on one hand, the mass flow controller can function autonomously and therefore with optimum precision, and, on the other hand, the supplementary electronic control of the fluid and delivery shut-off devices upstream and downstream from the flow controller allows the ozone or ozone/oxygen mixture amount to be delivered with particularly precise metering. This can be assisted by the additional embodiment of the invention, according to which one or more time intervals are generated in the electronic control system, during which the shut-off devices are opened to deliver the fluid based upon the predetermined metering.

With the mass flow controller, the set value for the fluid mass flow can be expediently adjusted externally via an interface. This can be implemented in a simple manner using a potentiometer, which can be adjusted manually.

For implementation of the invention, the mass flow controller for gases described on page 5 of the company publication “Massendurchflussmesser und-regler für Gase” [“Mass Flow Meters and Controllers for Gases”], published by M+W Instruments GmbH, D-85748 Garching, with a control valve integrated into the housing and a sensor made of noble steel, is suitable, for example.

The device is advantageously equipped with an ozone analyzer, with which the concentration of ozone in the fluid flow can be monitored and adjusted. This can be implemented expediently via the electronic control system. If the ozone concentration deviates from the set value, the therapy technician can detect this via an output from the electronic control system, and can react to it immediately and perform adjustments to ensure the safety and efficacy of the treatment.

Expanding upon this idea, the electronic control system is coupled with an ozone generator to allow its control via control engineering. By combining ozone generator and ozone analyzer, which are respectively monitored and/or controlled by the electronic control system, the ozone concentration in the fluid can be constantly controlled or adjusted to maintain the set value. If deviations should occur, the ozone generator can be actuated via the electronic control system using correspondingly adjusted parameters to produce an ozone concentration that corresponds to the set value.

In accordance with the invention, the mass flow controller, which is structurally inserted into the flow of fluid, ensures a constant volume or mass flow for the fluid mixture. For practical applications, set values of approximately 0.5-2.0 liters (corresponding to 0.7145-2.8858 grams) per minute have proven a particularly favorable compromise that will ensure uninterrupted treatment at a high comfort level.

Due to the very precisely controlled fluid mass flow in accordance with the invention, the timed control of the shut-off devices allows the amount of fluid delivered to be precisely metered. This purpose is promoted by a further embodiment of the invention, according to which the ozone generator is actuated and controlled during the time intervals or at a prevailing fluid flow in order to generate the most constant possible ozone quantity or mass per unit of time. Maintaining an ozone ratio of approximately 5-60 micrograms per milliliter ozone/oxygen mixture has proven practicable for this.

In accordance with one advantageous embodiment of the invention, the electronic control system monitors an actual value for the fluid mass flow, which value is provided by the mass flow controller via an interface. If the actual value exceeds threshold values which make normal or regular operation plausible, an alarm display or warning message is issued by the electronic control system to the operator (therapy technician). At the same time, the shut-off devices can be closed via the electronic control system to interrupt metering operation.

In accordance with an advantageous further improvement on the invention, a gas line pressure in the area of the metering outlet can be measured by one or more pressure sensors, and the measurement results can be detected at the outputs of the pressure sensors by the electronic control system, compared with one another by pressure sensors, which are redundantly arranged for reasons of safety, and displayed externally via a display or the like. In the case of intraperitoneal administration, the therapy technician can deduce the intraabdominal pressure from this and can draw conclusions as to the health status of the patient. In addition, the intraabdominal pressure prior to the start of administration of the ozone fluid is an important parameter in monitoring the absorption of the fluid introduced into the peritoneal cavity. This purpose is served by a further improvement of the described embodiment of the invention, in which the gas line pressure is stored or recorded in a memory device via the electronic control system. In the case of intraperitoneal administration, by determining the intraabdominal pressure the therapy technician can conclude when the administered fluid has been fully absorbed and therefore administration is completed.

In the case of intraperitoneal administration, as a safety measure to prevent the system from exceeding a previously established intraabdominal pressure, in accordance with one embodiment of the invention a pressure relief valve is provided, coupled to the electronic control system, which can open it. The latter is configured to open the pressure relief valve when the pressure sensor or sensors have determined that the threshold value for the gas line pressure, stored in advance in the electronic control system, has been exceeded.

For intraperitoneal administration of the ozone or ozone/oxygen mixture, the application set including a tube that creates a hollow space of substantial size, specified in greater detail below, is required. This tube expediently creates the connection between the metering outlet and the human or animal body via a cannula. This tube, along with any valves or bacterial filters (see below), must therefore be subjected to a washing process for cleaning and disinfection. For this purpose, in the case of one embodiment of the device according to the invention, a reverse flow washing intake for connection to the outlet of the ozone application device or the application set is provided adjacent to the metering outlet. Additionally, the metering outlet is configured for connection to the input of the application device. The electronic control system is configured in terms of programming and/or control engineering to actuate the two shut-off devices for fluid and delivery in order to clean the external application device via the metering outlet and the reverse flow washing intake. In this case, the administration tube is filled completely with ozone or ozone/oxygen mixture before actual administration into the human or animal body is begun. The advantage achieved thereby consists in that the ozone fluid is already present in the hollow cavity of the ozone administration device in a defined quantity, thereby ensuring metering precision.

Because ozone can represent a health hazard especially during inspiration, releasing surplus, ozonized fluid into the surrounding environment should be avoided. This condition is addressed by a further improvement of the device according to the invention in which one or more catalyzers are provided, which decompose surplus ozone, thereby adding to the operational safety of the device. This especially affects the ozone that is generated after the device is switched on until a stable ozone concentration is reached. This addresses the circumstance that, when the device is switched on (startup phase) certain fluctuations in the ozone concentration normally occur. Once a stable concentration has been established, the electronic control system can initiate administration via the metering outlet by opening the delivery/shut-off device.

The device according to the invention is intended for operating and/or for connection to a fluid source, such as a commercially available oxygen tank, for which at least one fluid source connection is provided. This connection can also be used to couple the device to a system for supplying medical oxygen, installed in a hospital, for example. To allow adjustment to respective pressure conditions, according to one embodiment, the device is equipped with a pressure relief valve, which is expediently arranged upstream from the intake of the mass flow controller or upstream from the fluid shut-off device. This pressure relief valve also allows the device of the invention to adjust to different connection standards.

Within the scope of the invention, the electronic control system can be provided with an interface for remote data transmission. This allows the control and monitoring software to be supplied with new updates.

Within the scope of the general inventive idea for achieving metering precision, an application set for connection to the metering outlet of the described device is proposed. The application set comprises a fluid line with a hollow cavity and two connection devices at the two ends, which are configured for coupling to the metering outlet and/or to the reverse flow washing intake of the device. To ensure that the ozone or ozone/oxygen mixture remains in the fluid line in a defined quantity once the washing process has been completed, a manually actuable shut-off element, such as a multiport plug valve, is inserted between the two connection devices. When the fluid line is separated from the reverse flow washing intake, the shut-off element, which has been closed manually in advance, prevents ozone from escaping, which would impair the precision of the planned metering. In order to ensure that no germs or bacteria are allowed to escape from the human or animal body into the device, a further embodiment of the invention provides for the application set to be equipped with a bacterial filter, which is inserted into the fluid line between the connection device allocated to the first outlet and the metering outlet and the shut-off element. Within the scope of the invention, the bacterial filter is especially configured such that even at low pressure levels, measurement of intraabdominal pressure will not be adulterated by a drop in pressure at the filter. In other words, the bacterial filter is configured for a minimum drop in pressure.

### BRIEF DESCRIPTION OF THE SEVERAL VIEWS OF THE DRAWINGS

Additional details, features, combinations of features, advantages, and effects based upon the invention are found in the following description of exemplary, preferred embodiments of the inventions, and from the set of drawings. The drawings show in:

FIG. 1, a plan view of the operating side of the metering device of the invention,

FIG. 2, a schematic fluid line flow plan for the device of FIG. 1,

FIG. 3, a schematic block diagram illustrating control engineering function,

FIG. 4, a schematic representation of the input-side piece of the application set,

FIG. 5, a schematic side view of the outlet-side piece of the application set, in a closed state,

FIG. 6, the outlet piece of FIG. 5 in its opened state,

FIG. 7, a plan view of the application set connected to the operating side of the device for reverse flow washing,

FIGS. 8a-8c, a flow chart representing the functioning of the metering system of the invention.

### DETAILED DESCRIPTION OF THE INVENTION

According to FIG. 1, the metering device identified as “Medozone IP” is equipped on its operating side with a color touch screen 65, above which are positioned an insufflation outlet 18 and a reverse flow washing intake 26, respectively configured as tubular connection ports.

This metering and administering device is used to prepare an ozone/oxygen mixture. Potential applications exist in hyperbaric ozone therapy, in collection via hypodermic needle, and in use as an outlet for permanent removal of the mixture. The illustrated exemplary embodiment of the invention represents an ozone therapy device for pneumoperitoneal insufflation (introduction of gas into the abdominal cavity). It insufflates a defined, very precisely metered quantity of ozone/oxygen mixture at a concentration of 5-60 μg/ml in multiple cycles. The patient's intraabdominal pressure is simultaneously monitored, and, once the patient's weight has been input via the touch screen, recommendations for treatment parameters are provided. Further, critical status indicators which may develop during the treatment are displayed to the therapy technician via the touch screen. Additionally, the most important treatment data are stored using the device software.

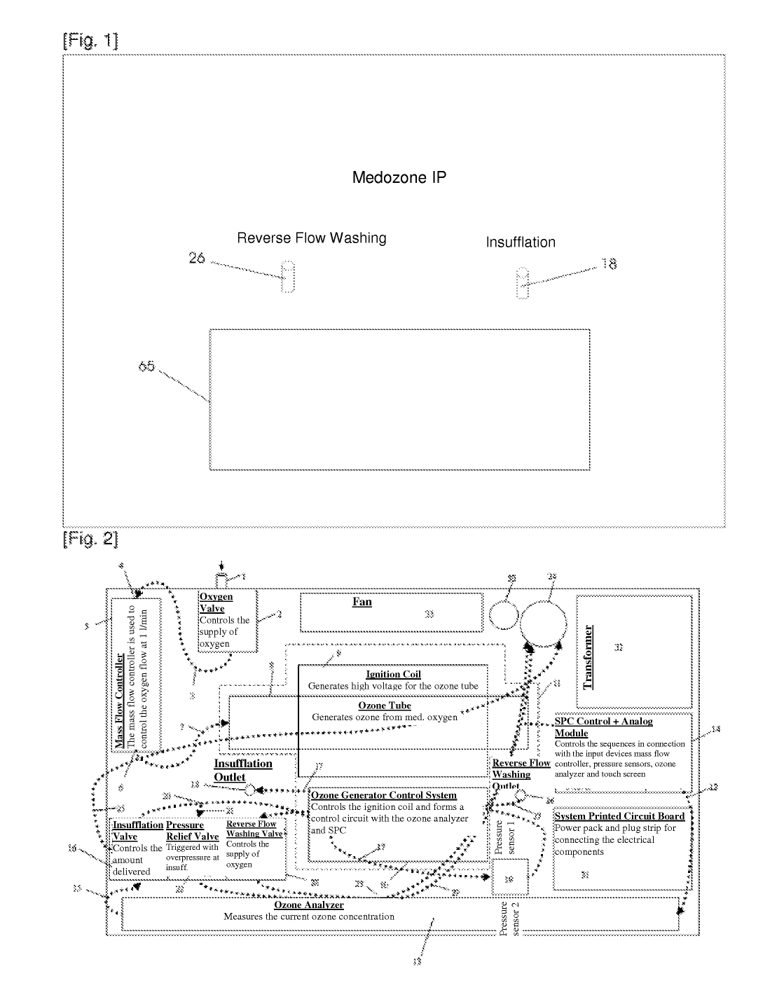

According to FIG. 2, the device is equipped at its input side with a fluid source connection 1, via which an oxygen source, for example a commercially available oxygen tank, can be connected. The connection 1 can be opened or closed using an oxygen valve 2, positioned immediately downstream from the connection, to control the supply of oxygen. A first connecting tube 3 (indicated by a dotted line) leads from the valve outlet to the intake 4 of a mass flow controller 5. This controller autonomously controls the oxygen mass flow or oxygen volume flow at one liter per minute with extreme precision. The fluid outlet 6 of the mass flow controller 5 is connected via a second connecting tube 7 to an ozone tube 8, which generates ozone in a known manner from the medical oxygen, which is supplied in a controlled manner via the oxygen valve 2. The high voltage required for this is generated by an ignition coil 9, which is controlled by the ozone generator control system 10. The ozone tube 8, the ignition coil 9 and the ozone generator control system 10 are elements of an ozone generator 11 (indicated by a dashed line). The outlet of the ozone tube 8 or the ozone generator 11 is connected via a third connecting tube 12 to an ozone analyzer 13, with which the current ozone concentration of the fluid mixture generated by the ozone generator 11 can be determined. The ozone concentration can thereby be continuously adjusted in a known manner, and can be controlled by means of an electronic control system 14, which is internal to the device. This control can expediently be implemented, for example, using a known stored program control (SPC) with integrated analog module (for example, digital/analog converter and analog/digital converter). The electronic control system 14 controls the sequences for input/output interfaces, for example touch screen, mass flow controller, pressure sensors (see below), ozone analyzer, and especially valves.

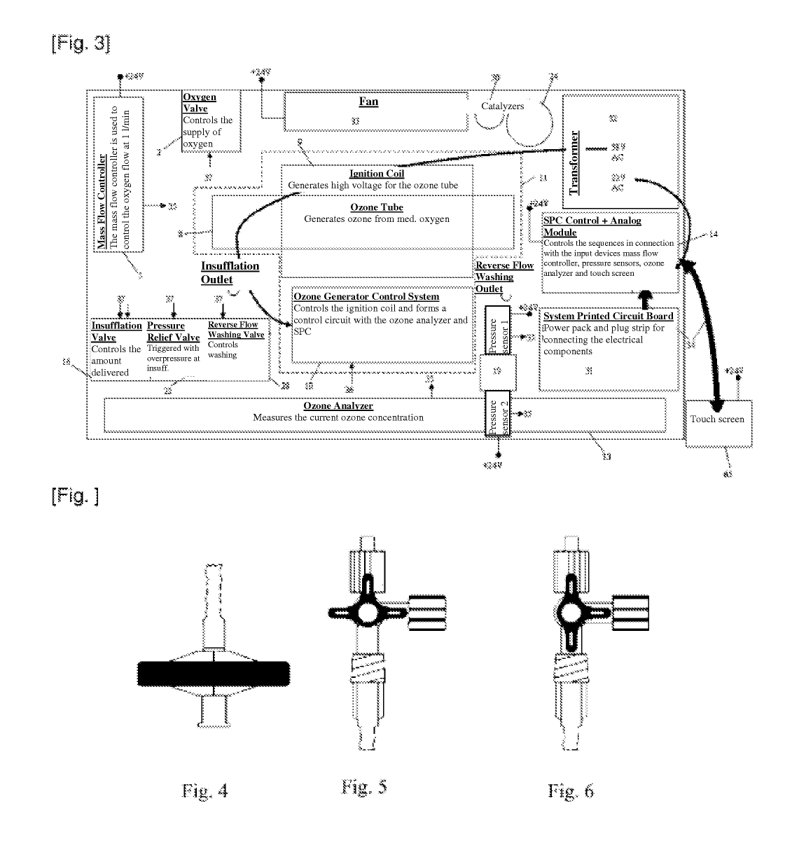

According to FIG. 2, the outlet of the ozone analyzer 13 is connected via a fourth connecting tube 15 to the intake of an insufflation valve 16 for controlling the amount of ozone or ozone/oxygen mixture that is delivered. At its outlet side, the insufflation valve 16, which constitutes the delivery/shut-off device described above, is connected via a fifth connecting tube 17 to an insufflation outlet 18, for connection to the application set described below. The fifth connecting tube 17 is functionally connected to a pressure sensor device 19. For safety reasons, this device can be implemented, for example, as redundant to a first and a second pressure sensor, as will be described in greater detail in reference to FIG. 3. In this manner, the pressure prevailing at the insufflation outlet 18 can be detected with a high degree of operational safety and reliability.

According to FIG. 2, the fifth connecting tube 17 is preferably equipped in the outlet area of the insufflation valve 16 with a branch line 20, from which a sixth connecting tube 21 leads to a pressure relief valve 22. When excess pressure is detected by the pressure sensor device, this valve is used to release ozone or ozone/oxygen mixture via a seventh connecting tube 23, connected at its outlet side, to a first decomposing catalyzer 24.

According to FIG. 2, the insufflation valve 16 is also equipped with a second outlet, from which an eighth connecting tube 25 also leads to the first decomposing catalyzer 24. This can be used to draw off and/or dispose of ozonized fluid which is generated during the startup phase of the device. The insufflation valve 16 is advantageously configured as a 3/2-way valve, which can optionally be switched via the electronic control means 14 to a closed state, to a state in which it is open to the insufflation outlet 18, or to a state in which it is open to the decomposing catalyzer 24.

According to FIG. 2, the device can optionally be equipped with a reverse flow washing intake 26, from which an eighth connecting tube 27 leads to an electronically actuable reverse flow washing valve 28. The ozone or ozone/oxygen mixture can thereby be selectively supplied during a possible application set washing phase (see below). The outlet of the shut-off valve 28 is also connected to the decomposing catalyzer 24 via a ninth connecting tube 29.

According to FIG. 2, a second, smaller decomposing catalyzer 30 is allocated to the catalyzer 24. The intake for this second decomposing catalyzer 30 is connected to the outlet of the decomposing catalyzer 24 and therefore receives the fluid that has already been largely cleaned of ozone by the decomposing catalyzer 24. Any ozone residues that may remain in the fluid are then decomposed in the catalyzer 30, so that the cleaned fluid can be delivered into the housing via small openings in the end of the decomposing catalyzer 30 that faces away from the fluid intake. The housing fan 33 then transfers the neutralized fluid together with the air in the interior of the housing to its exterior, so that the housing fan 33 also ensures the cooling of the device.

According to FIG. 3, in which the same reference symbols used in FIG. 2 are used to identify equivalent components or units, the device is equipped with a system printed circuit board 31, on which a power pack and a multiway connector for connecting the electrical units is provided. To supply the electronic control system 14 or the ozone generator 11 with alternating current, a transformer 32 is provided, which is coupled to the electronic control system 14 and/or to the ozone generator 11. A 24 V direct-current supply voltage can be drawn from the system printed circuit board or the power pack thereon for a wide range of device components, such as fan 33, electronic control system 14, touch screen 65, mass flow controller 5, pressure sensor system 19, ozone analyzer 13, etc. To enable a parallel exchange of data between the electronic control system 14 and the touch screen 65 and the system printed circuit board 31, multiwire data cables 34 are provided between these components.

According to FIG. 3, the two pressure sensors of the pressure sensor system 19, the ozone analyzer 13 and the mass flow controller 5 each supply an analog output signal 35, which can fluctuate between 0 V and +10 V, for example. To detect the analog outputs 35 of the various components, the electronic control system is equipped with the analog module (not shown), which can comprise one or more analog/digital converters, i.a., as input interfaces. The correspondingly digitalized signal values can then be further processed by the control and monitoring software within the electronic control system 14, as is described below in reference to the flow chart, for example. The analog module further comprises at least one output interface in the form of a digital/analog converter, with which an analog control signal 36 for controlling the ozone generator 11 in conjunction with the ozone analyzer 13 is sent to the ozone generator 11 or the ozone generator control system 10 (in a manner known from the company publication from the applicant, cited at the beginning of this application).

According to FIG. 3, all valves can be actuated using binary control signals 37, which are generated by the electronic control system in accordance with corresponding control software. Because the insufflation valve 16 is a multiway valve, which creates passage either to the decomposing catalyzer 24 or to the insufflation outlet 18, depending upon the prevailing startup phase or the initiated normal operating phase (for washing and insufflation), it is equipped with two binary control intakes 37.

According to FIG. 4, an application set has an intake-side piece 38, which is equipped with a fluid intake 39. This fluid intake is structured to complement the insufflation outlet 18 of the device of the invention, so that the intake piece 38 with the fluid intake 39 for the introduction of ozonized fluid can be placed on the insufflation outlet 18 to form a tight seal. The intake piece 38 of the application set has a housing 40, on which the fluid intake 39 is arranged. Inside the housing 40 is the bacterial filter 41. The bacterial filter 41 is configured as a membrane made of an ozone-proof material, preferably PTFE, Teflon, or noble steel. Membranes of this type have openings that are large enough to allow gas molecules such as oxygen and ozone to pass through, but represent an insuperable barrier to bacteria and germs. The micropores of the membrane have a pore diameter ranging from 20 to 90 μm, and with a pore diameter of 45 μm, a bacteria reduction of 99.9% is achieved. In this connection it is important for the pressure loss at the membrane to be minimized, to avoid affecting the measurement of intraabdominal pressure, which fluctuates within the range of a few tens of millibars. This is achieved by embodying the membrane with adequate surface area, ranging from 8 to 16 cm², or approximately 11.3 cm². Advantageously, this membrane can be situated in a housing having a round cross-section, especially with a low cylindrical shape. On the side of the housing 40 opposite the fluid intake 39, the connection device 38 has a fluid outlet 42, which is configured for connection to a flexible tube 43.

According to FIG. 5, the intake piece 38 is connected via the flexible tube 43 to the outlet piece 44 of the application set. The outlet piece of the application set has a fluid intake 45, a 2/3-way valve 46, a fluid outlet 47 for connection to the reverse flow washing intake 26 or a cannula/Braunula for administering the fluid into the abdominal cavity of the patient, and an additional outlet 48, which can be used for control purposes or for desufflating the patient. In this connection, fluid intake 45 and fluid outlets 47 and 48 can advantageously be embodied in accordance with the luer lock system. Using the 2/3-way valve 46, the flow of fluid at the outlet piece 44 can be adjusted as needed. When the 2/3-way valve 46 is in the position shown in FIG. 5 a connection is formed between outlets 47 and 48, however outlet 48 is closed by the screw cap 49. When the 2/3-way valve 46 is in this position, if outlet 48 is opened or if the screw cap 49 is removed, outlet 48 can be used for control or for desufflation. The fluid intake 45 is closed by the 2/3-way valve 46, so that no fluid is allowed to reach fluid outlets 47 and 48 from fluid intake 45.

In FIG. 6, the opened state is represented, in which fluid is allowed to reach both fluid outlet 47 and fluid outlet 48 from fluid intake 45. However, in FIG. 6, fluid outlet 48 is again closed by the screw cap 49, so that no fluid can escape from outlet 48.

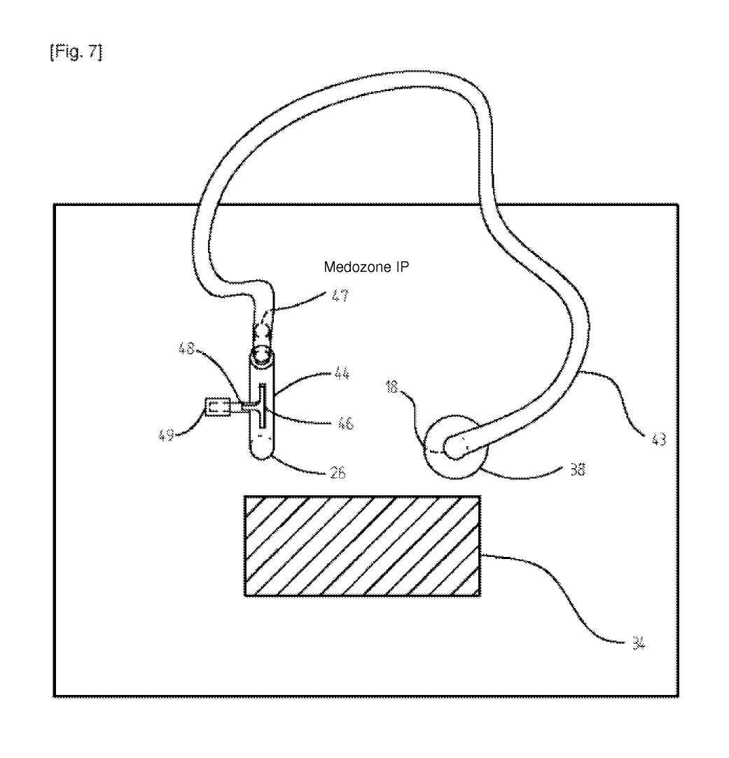

In FIG. 7, a plan view of the operating side of the device of the invention is shown, with an application set attached for reverse flow washing. The touch screen 65 is used for operation, in the case shown here to start the reverse flow washing process for the application set according to the invention. The application set is connected by its input piece 38, which contains the bacterial filter 41, to the insufflation outlet 18. The tube 34 is connected via the outlet piece 44 to the reverse flow washing intake 26, wherein the 2/3-way valve 46 is in the open position and fluid outlet 48 is closed by the sealing cap 49. Once the operator has started the reverse flow washing process using the touch screen 34, the insufflation valve 16 (see FIG. 2) is switched so that the produced ozone/oxygen mixture is conducted to the insufflation outlet 18. At the same time, the reverse flow washing valve 28 (see FIG. 2) is also switched, so that the connection to the catalyzer 24 (see FIG. 2) is opened, and in this manner, the ozone/oxygen mixture conducted from the insufflation outlet 18 via the application set and the reverse flow washing intake 26, is conducted through the reverse flow washing valve 28 to the catalyzer 24, where it is decomposed.

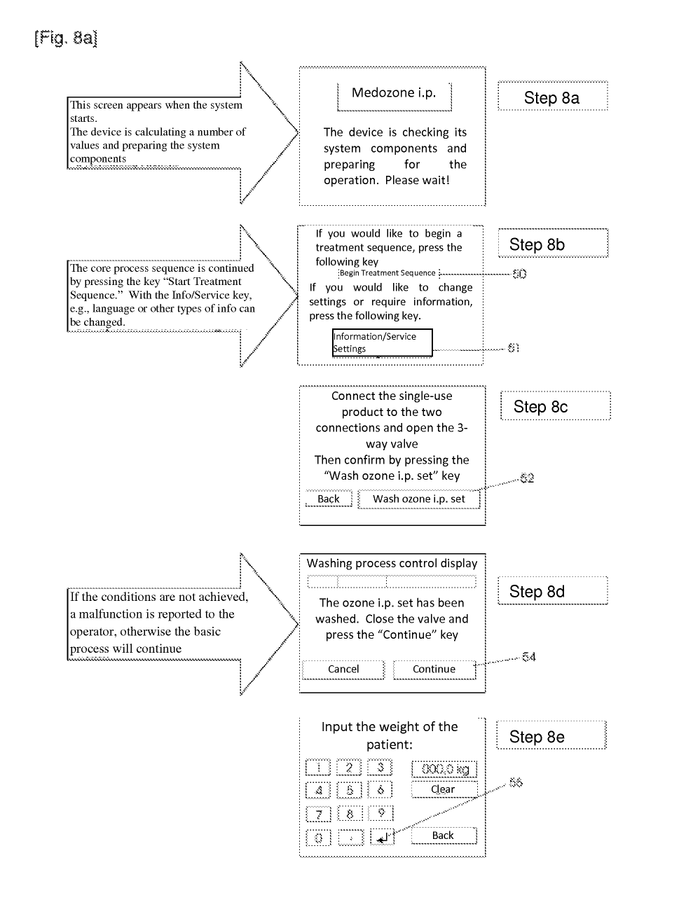

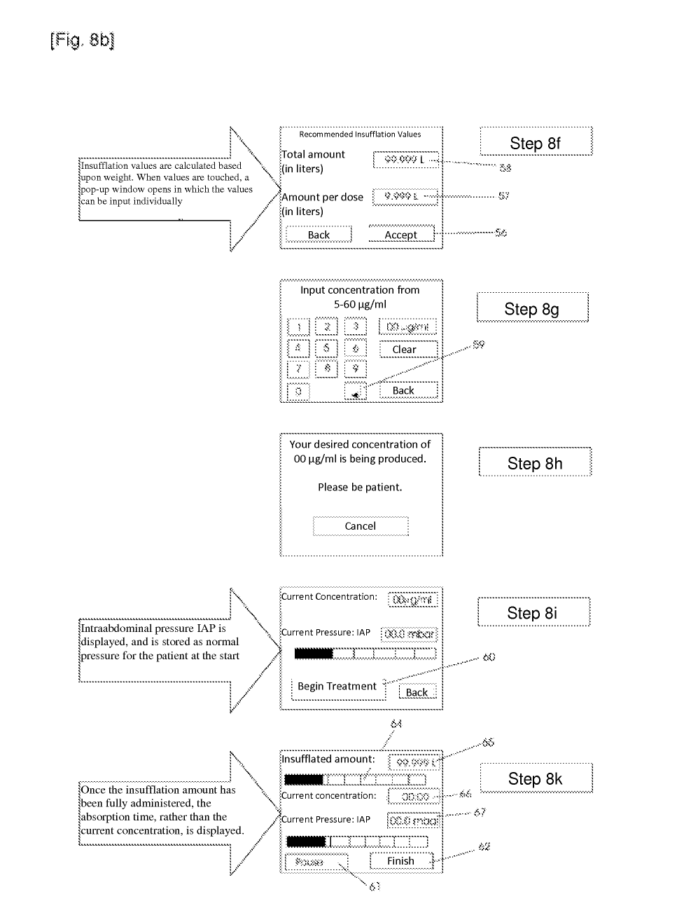

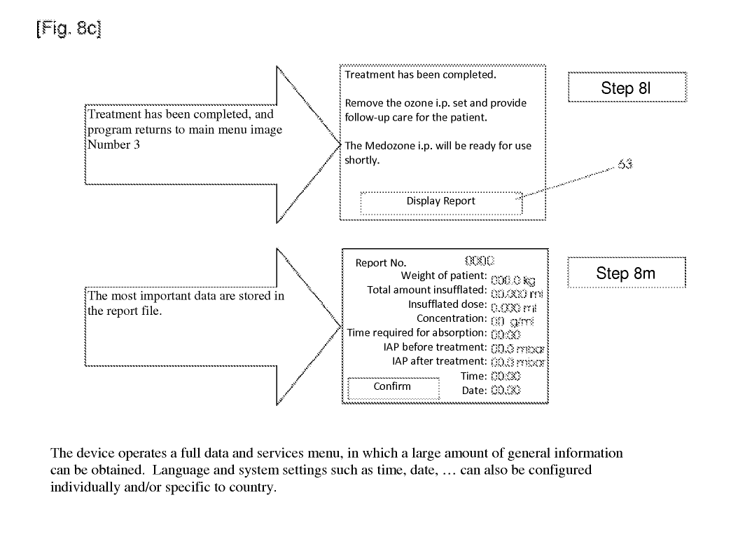

In FIGS. 8a-8c, the basic implementation of a treatment is illustrated within the framework of a flow chart comprising the steps 8a through 8m. In step 8a, the device performs a diagnostic routine, in which the functioning of the individual system components such as mass flow controller, ozone generator, ozone analyzer, pressure sensors and valves is tested and, if applicable, initialized. Once the diagnostics and initialization have been completed, in step 8b treatment can optionally be performed by pressing the key 50 on the touch screen 65, or the adjustment menu can be called up by pressing the key 51.

If a treatment sequence is selected, in step 8c the therapy technician is asked to connect the application set for reverse flow washing to the connections, and to open the 2/3-way valve. The therapy technician then attaches the application set with its intake piece 38 at the insufflation outlet 18 and with the outlet piece 44 at the reverse flow washing intake 26, and places the 2/3-way valve 46 in the position shown in FIG. 6. He then confirms the washing process by pressing the key 52.

In step 8d, the Medozone IP performs a washing process for the application set. The electronic control system 14 then opens the outlet of the insufflation valve 16, which is connected to the insufflation outlet 18, and also opens the reverse flow washing valve 28 to the catalyzer 24. He then starts the washing process by pressing the key 53. The oxygen valve 2 is opened by the electronic control system 14, and the mass flow controller 5 then conducts a quantity of fluid through the ozone generator 11 and the ozone analyzer 13 via the insufflation valve 16, the pressure sensors 19 and the insufflation outlet into the application set, wherein, after passing through the application set, the fluid is returned via the reverse flow washing intake 26 into the device, and is drawn off via the reverse flow washing valve 28 to the catalyzer 24. When the application set has been successfully washed, the electronic control system 14 reports this to the operator via the touch screen 65, and requests that he continue the treatment process by pressing the key 54.

In step 8e, the operator or therapy technician is then requested to input the weight of the patient using the touch screen 65. Once the patient's weight has been input using the number keys, and confirmed by pressing the key 55, the control system calculates a recommended insufflation amount in step 8f. This can still be adjusted by the operator by touching the values output in the output fields 57, 58 on the touch screen 65 and inputting different values in the pop-up window that opens up.

After confirming the insufflation amount by pressing the key 56, in step 8g the operator inputs the desired concentration for insufflation in μg/ml. Once this has been confirmed by the operator by pressing the key 59, the device begins to produce the desired concentration in step 8h. During this startup phase of production, the ozone concentration must still be regulated by the electronic control system 14 via the ozone generator 11, in that it performs a set value/actual value comparison using the actual ozone value measured in the ozone analyzer 13. For this set value/actual value comparison, when the oxygen valve 2 is opened the mass flow controller 5 conducts a constant mass flow through the ozone generator 11 and the ozone analyzer 13. The control of the ozone generator is adjusted accordingly via the electronic control system 14, until the set value for the concentration of ozone in the fluid is reached. In this connection, the ozone that is initially produced must still be decomposed safely and in an environmentally friendly manner. For this reason, during the startup phase the insufflation valve 16 is switched such that the outlet to the catalyzer 24 is open, thereby allowing the produced ozone to be drawn off to the catalyzer via the line 25.

Once a stable ozone concentration has been reached, the device signals readiness to start treatment in step 8i. The therapy technician can then start the treatment by pressing the key 60. The stored program control system 14 determines the intraabdominal pressure of the patient, stores this, and then begins to insufflate the planned amount. To accomplish this, the insufflation valve 16 is switched such that the ozonized fluid is conducted to the insufflation outlet 18 and there into the insufflation set. The electronic control system 14 monitors the current intraabdominal pressure using the pressure sensors 19, and in the event of overpressure opens the pressure relief valve 28, so that the ozonized oxygen will be drawn off to the catalyzer 24 if necessary. In step 8k, the insufflated amount is displayed by a progress bar 64 and an output field 65. The therapy technician also receives information on the current concentration and the current pressure in additional output fields 66, 67. If problems should occur, the therapy technician can pause or completely cancel the treatment by pressing the keys 61, 62. Once the total amount has been insufflated, the absorption time, rather than the current concentration, is displayed in the output field 66.

When the absorption time has expired, or when the therapy technician has manually ended follow-up monitoring by pressing the key 62, the device reports the successful completion of treatment in step 81, and asks the therapy technician to remove the application set. The therapy technician can then display a report by pressing the key 63, as is shown in step 8m. After displaying the report, the main menu in step 8b is called up again, and a new treatment sequence can be started.

### LIST OF REFERENCE SYMBOLS

1 Fluid source connection
2 Oxygen valve (fluid shut-off device)
3 First connecting tube
4 Mass flow controller intake
5 Mass flow controller
6 Mass flow controller outlet
7 Second connecting tube
8 Ozone tube
9 Ignition coil
10 Ozone generator control system
11 Ozone generator
12 Third connecting tube
13 Ozone analyzer
14 Electronic control system
15 Fourth connecting tube
16 Insufflation valve (delivery/shut-off device)
17 Fifth connecting tube
18 Insufflation outlet
19 Pressure sensor device
20 Branch line
21 Sixth connecting tube
22 Pressure relief valve
23 Seventh connecting tube
24 First decomposing catalyzer
25 Eighth connecting tube
26 Reverse flow washing intake
27 Eighth connecting tube
28 Reverse flow washing valve
29 Ninth connecting tube
30 Second decomposing catalyzer
31 System printed circuit board
32 Transformer
33 Fan
34 Data cable
35 Analog output
36 Analog input signal
37 Binary control signals
38 Input piece/connection device application set
39 Fluid intake intake piece application set
40 Housing intake piece application set
41 Bacterial filter/membrane
42 Fluid outlet input piece application set
43 Flexible tube application set
44 Outlet piece/connection device reverse flow washing application set
45 Fluid intake outlet piece application set
46 2/3-way valve
47 Fluid outlet outlet piece application set reverse flow washing/insufflation
48 Fluid outlet outlet piece application set control/desufflation
49 Sealing cap
50, 51, 52, 53, 54, 55, 56, 59, 60, 61, 52, 63 Keys touch screen
57, 58, 65, 66, 67 Input/output fields touch screen
64 Progress bar touch screen
65 Touch screen

1. Device for preparing and/or delivering ozone or an ozone/oxygen mixture in metered amounts from oxygen, having a fluid source connection (1) for providing oxygen, ozone or ozone/oxygen mixture and having a metering outlet (18)
characterized in that
a mass flow controller (5) is inserted in the fluid line from the fluid source connection (1) to the metering outlet (18), enabling the flow of fluid/mass to be controlled based upon a set value/actual value comparison, in that the intake of the mass flow controller (5) is functionally connected to or implemented as a fluid shut-off device (2), and the metering outlet (18) is functionally connected to or implemented as a delivery/shut-off device (16), and in that electronic control means (14) are connected at least to the two shut-off devices (2, 16) allowing them to be controlled via control engineering means.

2. Device in accordance with claim 1, characterized in that the control means (14) are equipped or configured in terms of program or control engineering so as to generate one or more intervals of time, during which the shut-off devices (2, 16) are actuated or actuable by the control means (14) for conducting fluid through the mass flow controller (5) and for delivering ozone or ozone/oxygen mixture through the metering outlet (18).

3. Device in accordance with claim 2, characterized in that the time interval or intervals are or can be preset via an input interface (65) of the control means (5) based upon a desired metering or delivery amount.

4. Device in accordance with claim 3, characterized in that a set value for a fluid mass flow can be adjusted manually in the mass flow controller (5).

5. Device in accordance with claim 3, characterized in that the fluid source connection (1) is connected to the intake (4) of the mass flow controller (5), to the output side (6) of which an ozone generator (8, 9, 10, 11) is connected downstream, the ozone generator being controllable via the control means (14) or actuable within the time interval or intervals for generating ozone from the supplied oxygen, and being connected at the output side at least indirectly to the metering outlet (18).

6. A device in accordance with claim 5, characterized in that the mass flow controller (5) is adjusted to a constant set value within the range of 0.7145 to 2.858 grams of oxygen per minute.

7. Device in accordance with claim 6, characterized in that the control means (14) are configured in terms of control or program engineering so as to actuate and control the ozone generator (8, 9, 10, 11) during the time intervals or at the prevailing oxygen mass flow, for generating a constant ozone quantity per unit of time.

8. Device in accordance with claim 7, characterized in that the ozone generator (8, 9, 10, 11) is actuated and controlled such that an ozone ratio of 5 to 60 micrograms per milliliter of ozone/oxygen mixture results.

9. Device in accordance with claim 2, characterized in that the control means (14) are connected to an output interface (35) of the mass flow controller (5) for monitoring an actual value for the oxygen mass flow, and are configured in terms of control or program engineering so as to control at least one of the shut-off devices (2, 16, 28) for the supply of oxygen for delivery via the metering outlet (18) or a touch screen output (65) for an error message, based upon the monitoring result.

10. Device in accordance with claim 9, characterized in that the control means (14) are connected at the input side to one or more pressure sensors (19), which are functionally connected to a gas line (17) which leads to the metering outlet, and the control means (14) are configured in terms of control or program engineering so as to detect, monitor, store, record, display on an output (65) a gas line pressure, using the pressure sensor signal or signals.

11. Device in accordance with claim 10, characterized in that the control means (14) are configured in terms of control or program engineering so as to determine, store and externally report the course of the gas line pressure over time.

12. Device in accordance with claim 11, characterized by a pressure relief valve (22) which can be actuated via the control means (14), and which is in functional connection with the delivery/shut-off device (16) or the metering outlet (18), and is connected at its outlet side to an outlet in the area around the exterior of the device, wherein the control means (14) are configured in terms of control or program engineering so as to cause the pressure relief valve (22) to open when the gas line pressure exceeds a threshold level stored in the control means.

13. Device in accordance with claim 12, characterized in that the pressure relief valve (22) is connected at its output side to the outlet via ozone decomposing means (24, 30).

14. Device in accordance with claim 13, characterized by a reverse flow washing intake (26) for connection to the outlet of an external ozone application device, wherein the metering outlet (18) is configured for connection to the intake of the application device, and the control means are configured in terms of program or control engineering so as to actuate the fluid and delivery shut-off device (2, 16) for washing the external application device via the metering outlet (18) and the reverse flow washing intake (26).

15. Device in accordance with claim 14, characterized by ozone decomposing means (24, 30), whereby the reverse flow washing intake (26) is connected to an outlet in the area around the exterior of the device.

16. Device in accordance with claim 15, characterized in that, in the direction of fluid flow, downstream from the reverse flow washing intake (26) a reverse flow washing shut-off device (28) is arranged and is connected to the control means (14) for its actuation, wherein said control means are configured in terms of program or control engineering so as to actuate the fluid, delivery and reverse flow washing shut-off devices (2, 16, 28), respectively opening these, simultaneously, for a predetermined washing time.

17. Device in accordance with claim 16, characterized in that a pressure relief valve is arranged upstream from the intake (4) of the mass flow controller (5) for connecting the device to a central oxygen supply or to an oxygen container.

18. Device in accordance with claim 17, characterized in that the mass flow controller (5) is configured as a separate structural unit with an internal control circuit, which functions independently of the electronic control means (14).

19. Device in accordance with claim 18, characterized in that the mass flow controller (5) has its own control valve.

20. A device in accordance with claim 1 and further comprising an application set for connection to the metering outlet (18) of the device, with a fluid line (43), with two ends (39, 47), each of which is equipped with a first or second device (38, 44) for connection to the metering outlet (18) or the reverse flow washing intake (26) of the device, characterized by a manually actuable shut-off element (46), which is inserted into the fluid line (43) between the two connection devices (38, 44).

21. A device in accordance with claim 20, characterized in that the shut-off element (46) is embodied as a multiport plug valve.

22. A device in accordance with claim 21 characterized in that the first or second connection device (38, 44) is embodied on the basis of the luer lock system.

23. A device in accordance with claim 22 characterized by a bacterial filter (41), which is inserted into the fluid line (43) between the first connection device (38) and the shut-off element (46).

24. A device in accordance with claim 23, characterized in that the bacterial filter (41) is produced with an ozone-proof membrane for a fluid operating pressure of less than 60 mbar.

25. A device in accordance with claim 24, characterized in that the bacterial filter (41) is structurally integrated with the first connection device (38) or the shut-off element (46) is structurally integrated with the second connection device (44).
